(19) United States
(12) Patent Application Publication
(10) Pub. No.: US 2022/0323723 A1
(43) Pub. Date: Oct. 13, 2022

(54) Catheter Placement System with Splittable Anchor

(71) Applicant: Bard Access Systems, Inc., Salt Lake City, UT (US)

(72) Inventors: Joe Spataro, Cottonwood Heights, UT (US); Daniel B. Blanchard, Bountiful, UT (US); Glade H. Howell, Draper, UT (US); Kyle G. Thornley, Farmington, UT (US)

(21) Appl. No.: 17/716,675

(22) Filed: Apr. 8, 2022

Related U.S. Application Data
(60) Provisional application No. 63/173,145, filed on Apr. 9, 2021.

(51) Int. Cl. A61M 25/06

(52) U.S. Cl. CPC A61M 25/0668; A61M 25/0693; A61M 25/065; A61M 2025/0675; A61M 25/09

(57) ABSTRACT

Embodiments of catheter placement systems described herein include a temporary splittable sheath anchor, disposed on an outer surface of a needle. The splittable anchor moves with respect to the needle when accessing the vessel, allowing for a temporary anchor to be created as soon as venous access is confirmed via flashback. The clinician can advance the anchor over the needle once vessel access has been confirmed by flashback. The needle can then be removed without further insertion into the vessel, mitigating accidental trauma. The guidewire can then be introduced through the anchor and advanced to a target location within the vasculature. The anchor can then be removed by splitting and withdrawing the anchor proximally. Embodiments further include a splittable advancement sheath including a guidewire and configured to be drawn thorough a housing and split to allow the guidewire to separate from the advancement sheath and advance into the vasculature.

### PRIORITY

This application claims the benefit of priority to U.S. Provisional Application No. 63/173,145, filed Apr. 9, 2021, which is incorporated by reference in its entirety into this application.

### SUMMARY

Briefly summarized, embodiments disclosed herein are directed to catheter placement systems with a splittable anchor sheath, and associated methods thereof. Conventional catheter placement techniques require the clinician to carefully stabilize the needle tip in the vein while feeding a guidewire through the needle. The guidewire serves to anchor the insertion site while the needle is removed and the catheter is inserted over the guidewire. However, this process requires the insertion and removal of multiple tools in order to place the catheter successfully. This results in a time consuming and complex process with an increased risk of introducing infection causing agents.

When placing elongate vascular access devices, such as central venous catheters (“CVC”) or the like, advanced insertion approaches are desirable. These advanced insertion systems and methods provide a simplified and expedited insertion processes while mitigating the introduction of infection causing agents. The plurality of components utilized are combined within a compact delivery device that maintains the tools within a sterile environment. Additionally, the insertion systems provide a soft, compliant anchor sheath that can be quickly placed within the target vessel once accessed, to stabilize the insertion site while the needle is removed.

Disclosed herein is a catheter placement system including, a housing, a flashback assembly slidably engaged with the housing, a needle supported by the flashback assembly, a catheter supported by a catheter advancement assembly that is slidably engaged with the housing, an anchor sheath slidably engaged with an outer surface of the needle, a portion of the anchor sheath configured to be advanced distally of the needle to anchor an incision site, and an anchor hub configured to split the anchor sheath axially and remove the anchor sheath proximally.

In some embodiments, the anchor sheath is formed of a compliant material including one of a plastic, polymer, PTFE, or an elastomer.

In some embodiments, the catheter includes one of a CVC catheter, a rapidly insertable central catheter, or a PICC catheter.

In some embodiments, the flashback assembly includes a syringe barrel in fluid communication with a lumen of the needle and a plunger slidably engaged therewith, the flashback assembly configured to draw a blood flow through the needle lumen.

In some embodiments, the catheter placement system further includes a guidewire disposed within a lumen of the catheter.

In some embodiments, the anchor hub includes an arm configured to engage a cleaver portion of the housing and configured to deflect the arm radially outward.

In some embodiments, the anchor hub includes a first arm coupled to a first portion of the anchor sheath and a second arm coupled to a second portion of the anchor sheath.

In some embodiments, the anchor sheath includes a tear line extending axially therealong and configured to allow the first portion of the anchor sheath to separate from the second portion of the anchor sheath.

In some embodiments, the tear line includes one of a groove, a score-line, a perforation, or a laser cut line.

Also disclosed is a method of placing a catheter including, accessing a vasculature with a distal tip of a needle and a distal tip of an anchor sheath assembly, the anchor sheath disposed on the needle, confirming vasculature access with a flashback assembly in fluid communication with the needle, sliding the distal tip of the anchor sheath distally of the distal tip of the needle into the vasculature, removing the needle proximally from the vasculature, advancing a guidewire through a lumen of the anchor sheath into the vasculature, splitting the anchor sheath along a longitudinal axis and removing the anchor sheath proximally, splitting a housing with a catheter advancement assembly to release a catheter therefrom, and advancing a catheter over the guidewire into the vasculature.

In some embodiments, splitting the anchor sheath includes withdrawing an anchor hub proximally over a cleaver portion, the cleaver portion splitting the anchor sheath along a tear line.

In some embodiments, the anchor hub includes a first arm coupled to a first portion of the anchor sheath, and a second arm attached to a second portion of the anchor sheath, each of the first arm and the second arm deflecting radially outward from a central axis to split the anchor sheath along the longitudinal axis.

In some embodiments, confirming vasculature access further includes withdrawing a plunger from a syringe barrel of the flashback assembly, and drawing a blood flow proximally through the needle and observing a blood flow.

In some embodiments, removing the needle proximally from the vasculature includes sliding the flashback assembly proximally, the needle coupled to the flashback assembly.

In some embodiments, advancing the catheter includes advancing a catheter advancement assembly supporting the catheter, the catheter assembly separating a first housing portion from a second housing portion to disengage the catheter from the housing.

Also disclosed is a catheter placement device including, a housing defining a housing lumen extending along a longitudinal axis and a channel extending at an angle relative to the longitudinal axis and intersecting the housing lumen, a needle supported by a syringe and configured to extend through the housing lumen, an anchor sheath supported by an anchor hub that is coupled to a distal end of the housing, the anchor sheath slidably engaged with the needle, and a catheter including a guidewire disposed within a lumen of the catheter and contained within an advancement sheath, a distal end of the advancement sheath configured to extend through the channel, one or both of the guidewire and the catheter configured to separate from the advancement sheath within the channel and extend through the housing lumen.

In some embodiments, the advancement sheath includes a tear line extending along an axial length and configured to allow a first portion of the advancement sheath to separate from a second portion of the advancement sheath to allow one of the guidewire or the catheter to separate from the advancement sheath.

In some embodiments, the housing further includes a blade disposed proximate a junction between the channel and the housing lumen and configured to cut through a wall of the advancement sheath as the advancement sheath is urged through the channel.

In some embodiments, the anchor hub includes a separation line configured to facilitate separation therealong and to split the anchor sheath along a tear line extending axially along the anchor sheath.

In some embodiments, the anchor hub further includes a first wing and a second wing hingedly rotatably relative to each other and configured to separate a first portion of the anchor hub from a second portion of the anchor hub.

Also disclosed is a method of placing a catheter including, accessing a vessel with a needle and anchor sheath assembly supported by a housing, advancing a distal tip of the anchor sheath distally of a distal tip of the needle, into the vessel, withdrawing the needle from the vessel, moving an advancement sheath through a channel of the housing to advance a guidewire disposed within the advancement sheath, through a lumen of the anchor sheath and into the vessel, splitting the anchor sheath, withdrawing the anchor sheath proximally, and advancing a catheter over the guidewire and into the vessel.

In some embodiments, the lumen of the housing extends along a longitudinal axis and the channel extends at an angle relative to the longitudinal axis and intersects the housing lumen.

In some embodiments, advancing the catheter includes pulling the advancement sheath over the insertion site, the guidewire splitting the advancement sheath and urging the catheter disposed therein over the guidewire and into the insertion site.

In some embodiments, the advancement sheath includes a tear line extending along an axial length and configured to allow a first portion of the advancement sheath to separate from a second portion of the advancement sheath to allow one or both of the guidewire and the catheter to separate from the advancement sheath.

In some embodiments, the housing further includes a blade disposed proximate a junction between the channel and the housing lumen and configured to cut through a wall of the advancement sheath as the advancement sheath is urged through the channel.

In some embodiments, the method further includes an anchor hub supporting the anchor sheath and including a separation line configured to facilitate separation therealong and to split the anchor sheath along a tear line extending axially along the anchor sheath.

In some embodiments, the anchor hub further includes a first wing and a second wing hingedly rotatably relative to each other and configured to separate a first portion of the anchor hub from a second portion of the anchor hub.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a catheter placement system including a splittable anchor sheath, in accordance with embodiments disclosed herein.

FIG. 1B shows a perspective underside view of the catheter placement system of FIG. 1A, in accordance with embodiments disclosed herein.

FIGS. 2A-2C show close up perspective views of the splittable anchor and flashback assembly of the catheter placement system of FIG. 1A, in accordance with embodiments disclosed herein.

FIGS. 3A-3E show an exemplary method of use of a catheter placement system, in accordance with embodiments disclosed herein.

FIGS. 4A-4C show an exemplary method of splitting a splittable anchor, in accordance with embodiments disclosed herein.

FIG. 4D shows a cross-sectional view of the splittable anchor, in accordance with embodiments disclosed herein.

FIGS. 5A-5B show an exemplary method of splitting a housing of a catheter placement system, in accordance with embodiments disclosed herein.

FIGS. 6A-6B show perspective views of a catheter placement system including a splittable anchor and a splittable advancement sheath, in accordance with embodiments disclosed herein.

FIG. 6C shows details of the housing of the catheter placement system of FIGS. 6A-6B, in accordance with embodiments disclosed herein.

FIGS. 6D-6E show cross-section views of the catheter placement system of FIGS. 6A-6B, in accordance with embodiments disclosed herein.

FIGS. 7A-7B show cross-section views of the splittable anchor of the catheter placement system of FIGS. 6A-6B, in accordance with embodiments disclosed herein.

FIGS. 8A-8H show an exemplary method of use of a catheter placement system, in accordance with embodiments disclosed herein.

FIG. 9 shows a cross-section view of an advancement sheath and catheter accessing a vessel, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

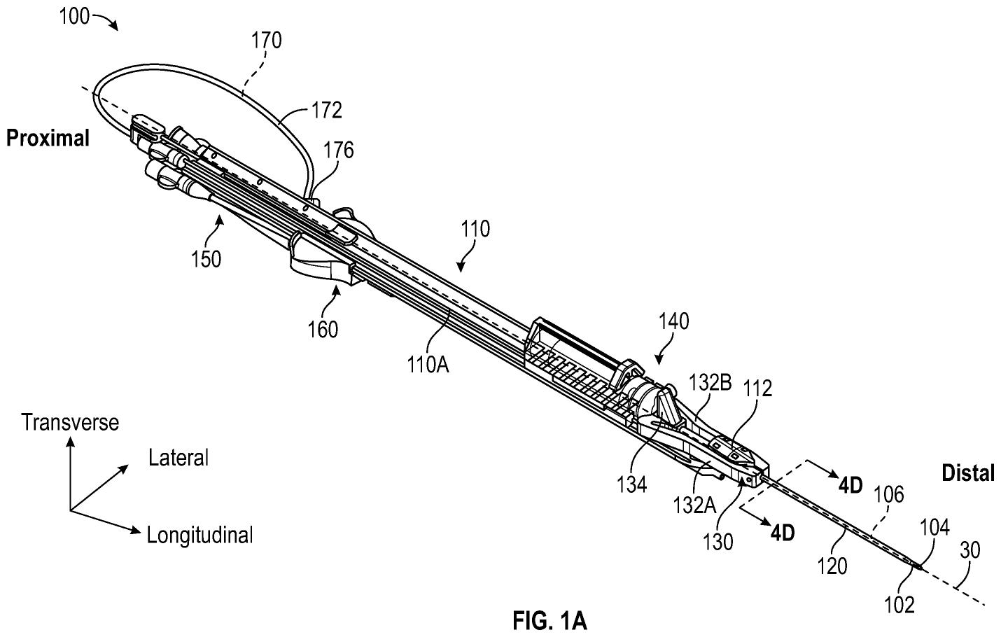

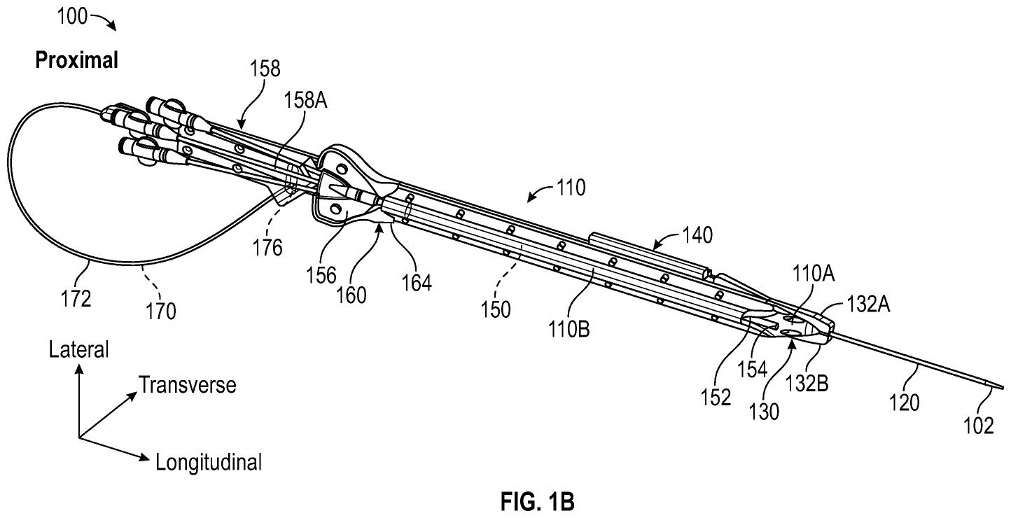

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

Embodiments described herein, use a temporary splittable sheath anchor, disposed on an outer surface of the needle. The splittable anchor moves with respect to the needle when accessing the vessel, allowing for a temporary anchor to be created as soon as venous access is confirmed via flashback. The clinician can advance the anchor over the needle once vessel access has been confirmed by flashback. The needle can then be removed without further insertion into the vessel, mitigating accidental trauma or “backwalling,” the vessel with the needle. The guidewire can then be introduced through the anchor and advanced to a target location within the vasculature. The anchor can then be removed by splitting and withdrawing the anchor proximally.

Advantageously, the splittable anchor can be placed immediately once vascular access is confirmed by flashback and contrasts with conventional insertion techniques that advance a guidewire anchor subsequent to vascular access confirmation. Further, the splittable anchor sheath can allow for early removal of the needle in the insertion process, mitigating the risk of vessel damage due to accidental movement of the needle or from over advancement of the needle resulting in “backwalling” the vessel. As used herein, the term “backwalling” is where a needle is advanced to access a vessel and subsequently advanced through a far wall of the vessel, resulting in various complications.

Advantageously, the splittable sheath anchor is disposed on an outer surface of the needle providing a clear pathway for the guidewire and/or catheter to advance into the vessel. This can minimize repeated insertion and removal of multiple tools, expediting the placement process and mitigating the introduction of pathogens or similar infectious causing agents. Further, the entire system can be contained and operated within a sterile environment, preventing exposure and reducing the risk of contamination. Advantageously, a distal portion of the sheath anchor can align an axis of the guidewire passing therethrough, with an axis of the vessel, further mitigating backwalling of the vessel during insertion of the guidewire.

FIGS. 1A-1B show perspective views of a catheter insertion system (“system”) 100. FIG. 1A shows an upper side perspective view of the system 100. FIG. 1B shows an under side perspective view of the system 100. The system 100 can generally include a housing 110, a needle 102, and a splittable anchor 120 disposed on an outer surface of the needle 102 and supported by an anchor hub 130. The system 100 can further include a flashback assembly 140, a catheter 150 supported by a catheter advancement assembly 160 and a guidewire 170.

The needle 102 can extend along a longitudinal axis and include a sharpened distal tip 104 configured for piercing a skin surface 70 and accessing a vasculature of a patient. The needle 102 can define a needle lumen 106 configured to provide fluid communication between the distal tip 104 and a flashback assembly 140. In an embodiment, a proximal end of the needle can be supported by the flash back assembly 140. In an embodiment, the needle 102 can be coupled to the flashback assembly 140 by a conduit 146, formed of a transparent material.

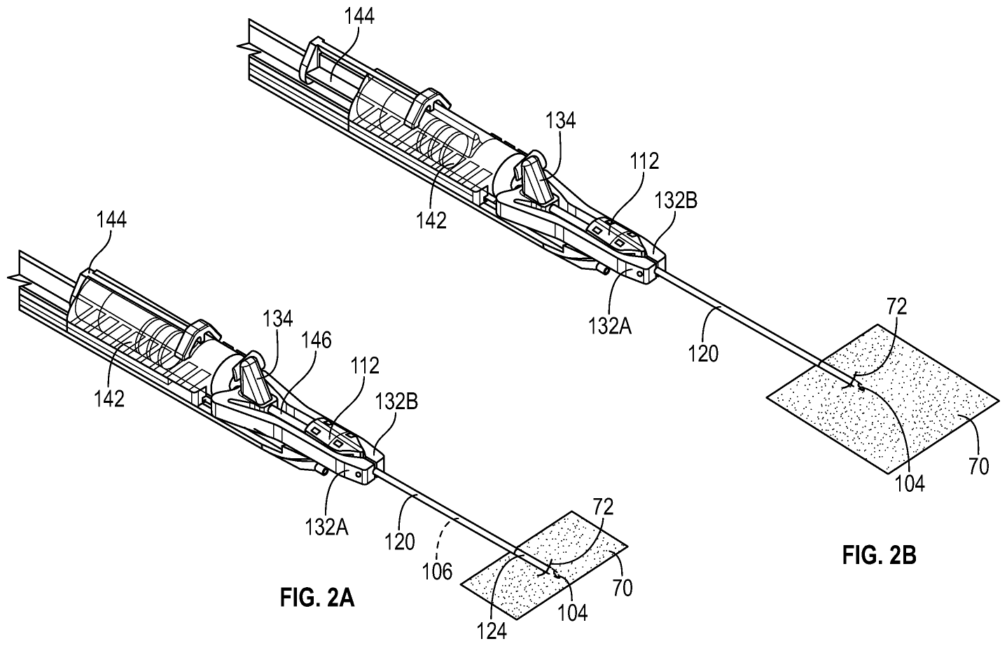

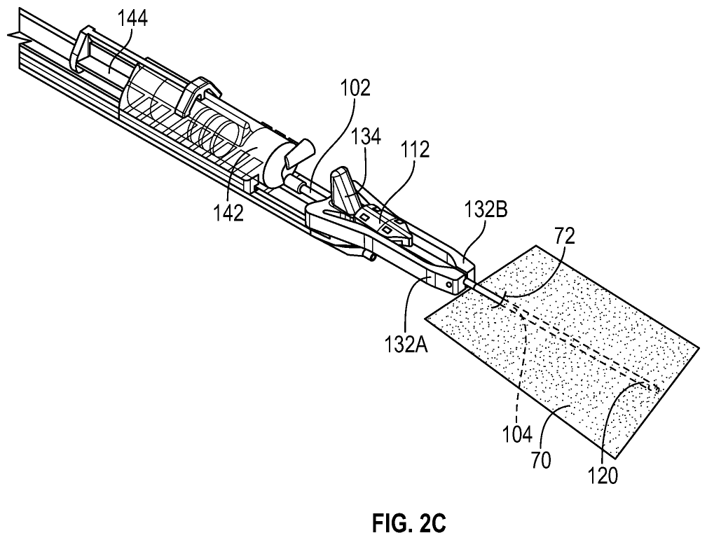

As shown in more detail in FIGS. 2A-2C, the flashback assembly 140 includes a syringe barrel 142 coupled to a proximal end of the needle 102 and in fluid communication with a needle lumen 106. The syringe barrel 142 can define a cavity and include a plunger 144 slidably engaged therewith. As shown in FIGS. 2A-2B, sliding the plunger 144 proximally relative to the syringe barrel 142 can create a vacuum therein, which can cause a proximal fluid flow through the needle lumen 106 and into to the syringe barrel 142. In an embodiment, a portion of the syringe barrel 142 or a portion of the conduit 146 disposed between the needle lumen 106 and the syringe barrel 142, can be formed of a transparent material configured to allow observation of a blood flow therethrough. As such a clinician can observe a color and/or pulsatile flow to confirm correct vascular access. In an embodiment, the syringe barrel 142 can be slidably engaged with the housing 110 along a longitudinal axis. Sliding the syringe barrel 142 proximally can cause the needle 102, coupled thereto to slide proximally relative to the housing 110.

With continued reference to FIGS. 1A-1B, the system 100 can further include a splittable anchor 120 disposed annularly on an outer surface of the needle 102 and slidably engaged therewith. The anchor 120 can define a substantially elongate tubular shape with a tapered distal tip 124. The distal tip 124 can fit tightly about the needle 102 to allow the needle 102 and anchor 120 assembly to be inserted through an insertion site 72 and into a vasculature of a patient. In an embodiment, the anchor 120 can be formed of a soft, compliant material such as a plastic, polymer, elastomer, or the like.

In an embodiment, the anchor 120 can be supported at a proximal end by an anchor hub 130 configured to slide the anchor 120 relative to the needle 102, as described in more detail herein. In an embodiment, the anchor hub 130 can include a finger pad 134 configured to facilitate manipulation of the anchor hub 130 and anchor 120 coupled thereto, by the clinician. In an embodiment, a distance between the distal tip 104 of the needle 102 and the distal end of the anchor hub 130 can be between 5 cm and 10 cm long. However, it will be appreciated that greater or lesser distances are also contemplated. In an embodiment, a distance between the distal tip 104 of the needle 102 and the distal end of the anchor hub 130 can be 7 cm long.

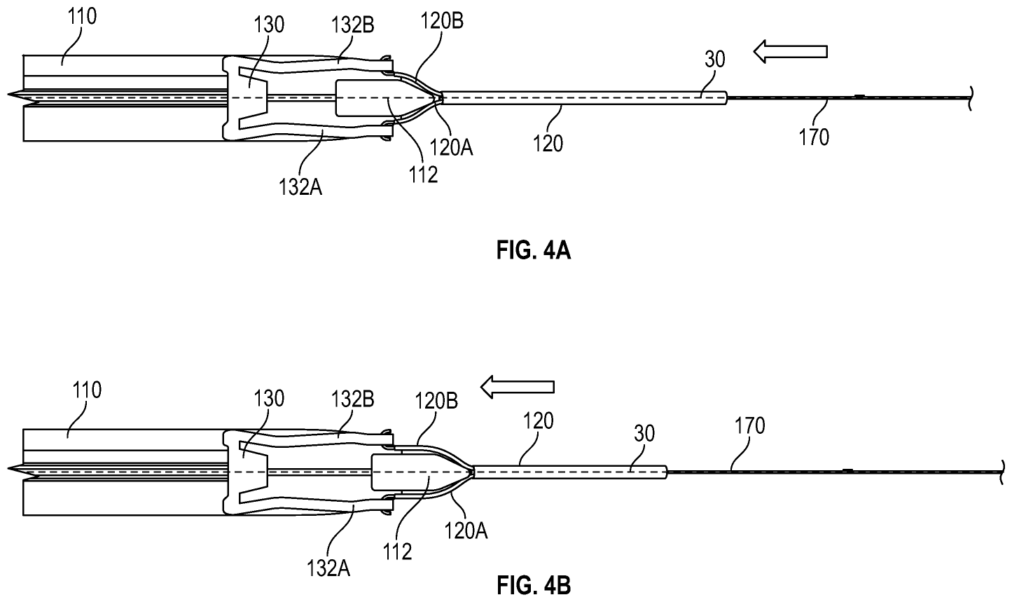

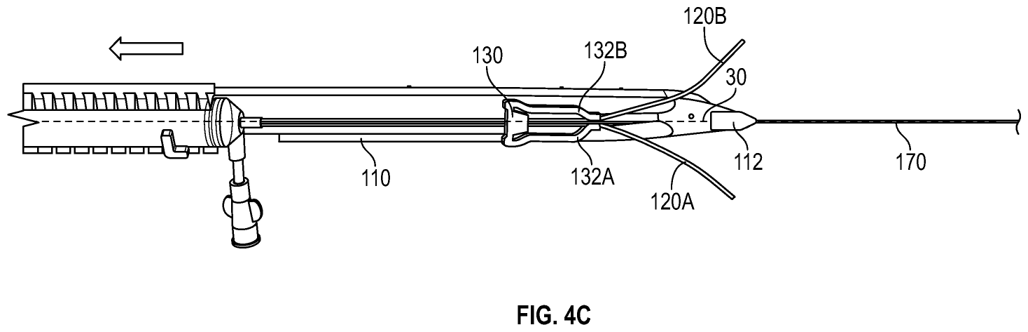

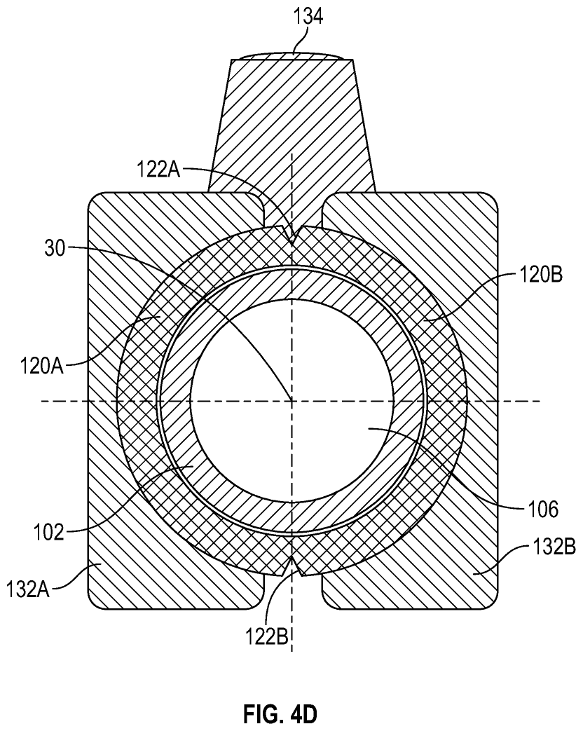

In an embodiment, as shown in FIG. 4D, the splittable anchor 120 can include one or more tear lines 122 extending longitudinally along an outer surface thereof. The tear line 122 can include a groove, score-line, perforation, laser cut line, or similar line of weakness extending longitudinally and extending into a wall of the anchor 120. The tear line 122 can be configured to facilitate separation of two or more portions of the anchor 120 to allow removal of the anchor 120. In an embodiment, the anchor 120 can include a first tear line 122A and a second tear line 122B disposed opposite each other across a central axis 30 of the needle 102 and configured to allow separation of the anchor 120 into two longitudinal halves. However, it will be appreciated that other numbers and configurations of tear lines 122 are also contemplated to fall within the scope of the invention.

In an embodiment, the anchor hub 130 can be slidably engaged with the housing 110 and configured to either advance the anchor distally, or withdraw the anchor 120 proximally. In an embodiment, the anchor hub 130 can include an arm 132 extending distally therefrom. A distal portion of the arm 132 can be coupled with a proximal portion of the anchor 120, supporting the anchor 120. As shown, the anchor hub 130 can include a first arm 132A coupled to a first portion 120A of the anchor 120 and a second arm 132B coupled to a second portion 120B of the anchor 120. However, it will be appreciated that other numbers and configurations of anchor hub arms 132 are also contemplated. Urging the anchor hub 130 proximally can cause the first arm 132A and the second arm 132B to separate radially outwards from the central axis 30 of the needle, splitting the anchor 120 along a tear line 122.

As shown in FIGS. 2A-2C and FIGS. 4A-4C, in an embodiment, the housing 110 can include a cleaver 112 disposed between the first arm 132A and the second arm 132B of the anchor hub 130 and defining a wedge-shaped distal edge. The cleaver 112 can be configured to separate the first arm 132A and the second arm 132B radially outwards as the anchor hub 130 is urged proximally. In an embodiment, the distal edge of the cleaver 112 can align with the tear line 122 and can be configured to separate the anchor 120 along the tear line 122. In an embodiment, the cleaver 112 can include a sharpened distal edge, and/or can include a blade, configured to cut the anchor 120 along a longitudinal axis, as the anchor 120 is withdrawn proximally. Advantageously, the cleaver 112 can be configured to reduce any friction or drag during the splitting process.

With continued reference to FIGS. 1A-1B, in an embodiment, the system 100 can further include a catheter 150, supported by a catheter advancement assembly 160. As shown, the catheter 150 can be a multi-lumen central venous catheter (“CVC”). However, it will be appreciated the CVC catheter 150 is a non-limiting example, and that embodiments disclosed herein can be used with various single lumen or multi-lumen, elongate vascular access devices such as peripherally inserted central catheter (PICC), rapidly insertable central catheters (RICC), dialysis catheters, and the like, without limitation.

The catheter 150 can generally include an elongate body 152 extending along a longitudinal axis and defining one or more catheter lumen. A distal tip 154 of the catheter body 152 can include an opening communicating with a lumen. A proximal end of the body 152 can be supported by a catheter hub 156 that can be releasably engaged with the catheter advancement assembly 160. In an embodiment, the catheter 150 can further include one or more extension legs 158 extending proximally from the catheter hub 156. Each extension leg can communicate with a lumen of the catheter body 152. In an embodiment, the catheter advancement assembly 160 can be releasably engaged with one or more portions of the catheter 150, e.g. a catheter hub 156, extension leg 158, catheter body 152, or the like. The catheter advancement assembly 160 can be slidably engaged with the housing 110 and can be configured to advance the catheter 150 distally relative to the housing 110.

In an embodiment, the housing 110 can include a first, upper portion 110A and a second, lower portion 110B releasably engaged with each other and configured to contain a portion of the catheter 150, e.g. the catheter body 152 or the like, therebetween. In an embodiment, as the catheter advancement assembly 160 is urged distally relative to the housing 110, a wedge-shaped distal portion 164 of the catheter advancement assembly 160 can be urged between the first portion 110A and the second portion 110B of the housing 110, separating the housing 110 and releasing the catheter 150 therefrom.

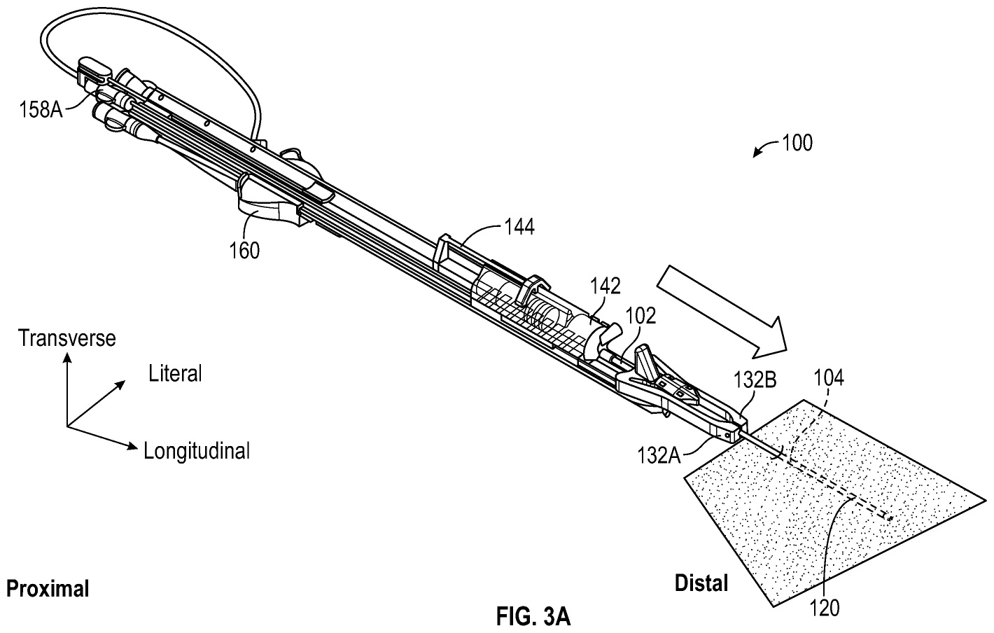

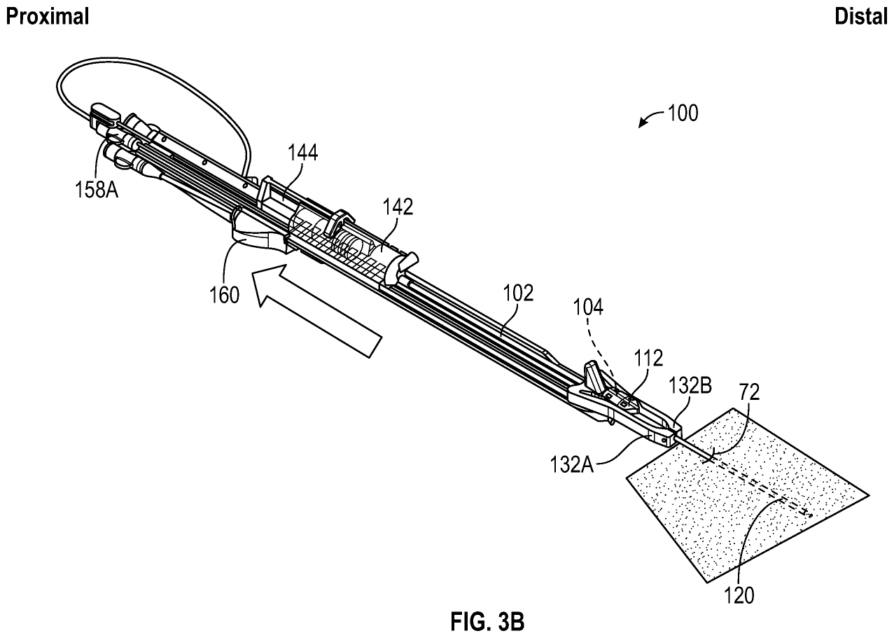

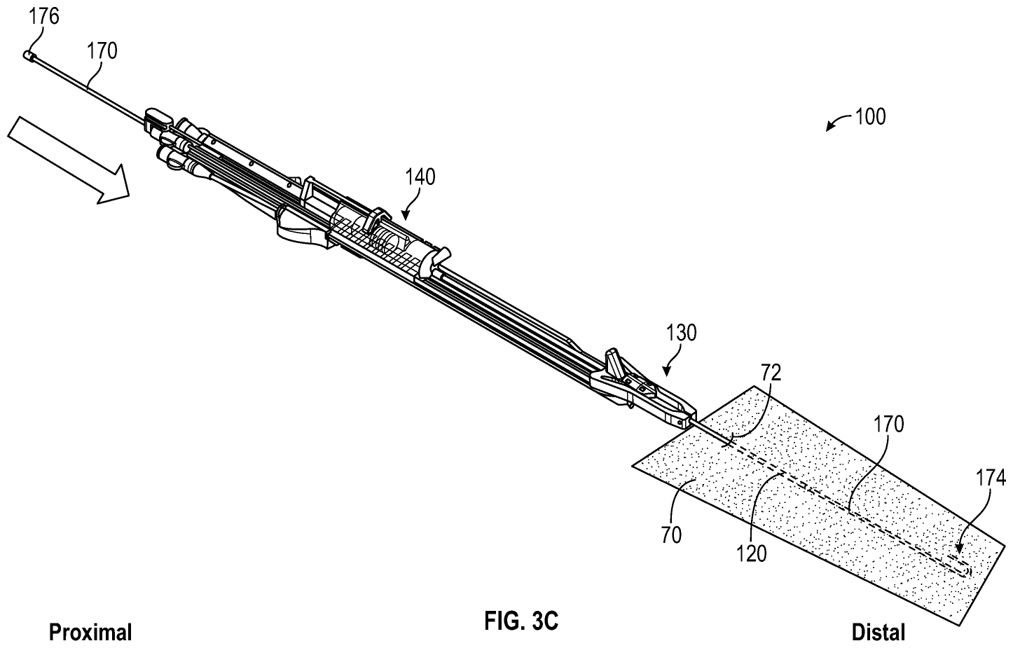

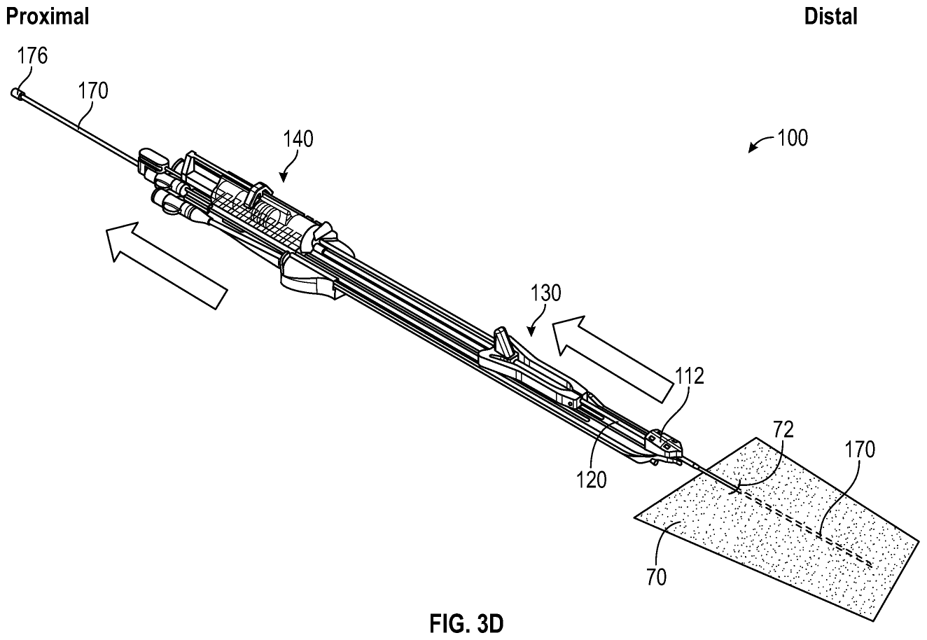

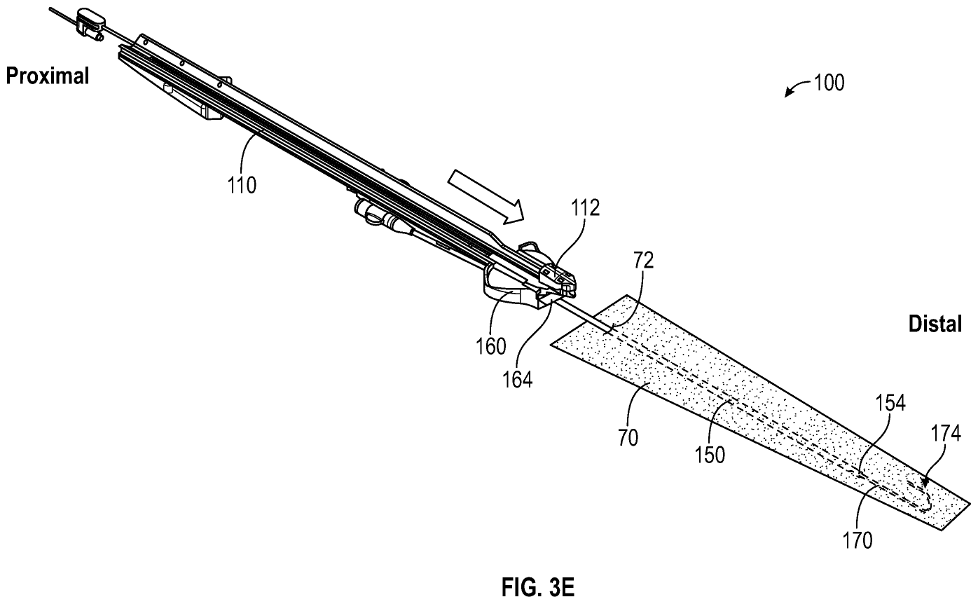

In an embodiment, the system 100 can further include a guidewire 170. In an embodiment, the guidewire 170 can be “preloaded” within a lumen of the catheter 150 where a distal tip 174 of the guidewire 170 can extend through a first extension leg 158A, through the catheter hub 156 and through a lumen of the catheter body 152. A proximal portion of the guidewire 170 can extend proximally of the catheter 150 and can be contained within a guidewire sheath 172. In an embodiment, a proximal end 176 of the guidewire 170, disposed within the guidewire sheath 172 can be recurved and coupled with a portion of the housing 110 at a convenient angle. The guidewire 170 can be slidably engaged with the lumen of the catheter 150 and can be advanced distally such that a distal tip 174 can advance distally of a distal tip 104 of the needle 102. Advantageously, recurving the guidewire 170 back onto the housing 110 maintains the system 100 within a compact space and prevents the proximal end of the guidewire 170 from obstructing the clinician during use. In an embodiment, as shown in FIG. 3C, a proximal end 176 of the guidewire 170 can include a guidewire hub, or similar structure, permanently coupled thereto and configured to prevent the proximal end 176 from passing through a lumen of the catheter 150 and into the vasculature of the patient.

In an embodiment, as shown in FIG. 3E, a distal tip 174 of the guidewire 170 can include a “coil tip” configured to mitigate trauma to the vessel as the guidewire 170 is advanced therethrough. The “coil tip” 174 can define a non-linear configuration in a relaxed state but can be elastically deformed to a linear configuration when disposed within the catheter 150 and/or anchor 120 lumens. As such, as the guidewire distal tip 174 is advanced distally of the anchor 120, the distal tip 174 can resume the relaxed state configuration of a non-linear, bent or coiled shape, providing an increased surface area to a distal most surface of the guidewire 170 and can prevent puncturing the vessel wall as the guidewire 170 is urged through the tortuous pathway to a target location within the vasculature.

In an exemplary method of use, as shown in FIGS. 2A-3E, a catheter placement system 100 with a splittable anchor 120 is provided, as described herein. As shown in FIG. 2A, a distal tip 104 of the needle 102 can puncture a skin surface 70 and the distal tip 104 of the needle 102 and a distal tip of the anchor 120 can access a vasculature of a patient. A blood flow can flow proximally through the needle lumen 106 and through a transparent conduit 146. A clinician can then observe a color and/or pulsatile flow to confirm correct vascular access. In an embodiment, as shown in FIG. 2B, a clinician can withdraw the plunger 144 proximally to create a vacuum within the syringe barrel 142 and draw a blood flow proximally through the conduit 146 and into the syringe barrel 142. In an embodiment, a portion of the syringe barrel 142 can be transparent to allow a clinician to observe a color and/or a pulsatile flow.

As shown in FIG. 2C, once vascular access has been confirmed, a clinician can then advance the anchor hub 130 distally to advance the splittable anchor 120 into the vasculature of the patient. It is important to note that only a tip 104 of the needle 102 need enter the vasculature and confirm access by flashback, after which the soft, compliant anchor sheath can be advanced into the vasculature to anchor the insertion site 72. In an embodiment, the anchor 120 can be advanced by approximately 2 cm, however, greater or lesser advancement distances are also contemplated. Advantageously, the anchor 120 can be formed of a soft compliant material and can mitigate any trauma to the vessel as the anchor 120 is advanced. Exemplary materials forming the anchor 120 includes plastic, polymer, elastomer, natural rubber, latex, synthetic rubber, silicone rubber, polytetrafluoroethylene (PTFE), expanded polytetrafluoroethylene (ePTFE), or the like.

As shown in FIGS. 3A-3B, with the anchor sheath 120 in the distal, anchored configuration, anchoring the insertion site 72, the needle 102 can then be withdrawn proximally. In an embodiment, the flashback assembly 140 that is supporting the needle 102 can then be withdrawn proximally. In an embodiment, the syringe barrel 142 that is slidably engaged with the housing 110 can be slid proximally relative to the housing 110 to withdraw the needle tip 104 from the vasculature of the patient or withdraw the needle 102 from the anchor 120 entirely. In an embodiment, the needle 102 can be withdrawn until the needle tip 104 is disposed within a safety mechanism configured to prevent needle stick injuries. In an embodiment, the safety mechanism can be disposed within the cleaver portion 112 of the housing 110. As such the needle 102 can be removed from the vasculature and safetied while the anchor maintains patency of the insertion site 72.

As shown in FIG. 3C the proximal end 176 of the guidewire 170 can then be released from the housing 110, from the recurved position to resume a linear configuration in a relaxed state. The clinician can then manipulate the proximal end 176 of the guidewire 170 to advance the distal tip 174 of the guidewire 170 through the lumen of the catheter 150, through the anchor 120 that is maintaining patency of the insertion site 72 and into the vasculature of the patient until the distal tip 174 is disposed at a target location within the vasculature.

In an embodiment, the guidewire sheath 172 disposed between the proximal end 176 of the guidewire 170 and the proximal end of the first extension leg 158A, can be formed of a thin material and can be collapsible to allow the guidewire 170 to be advanced while preventing the clinician from directly contacting the guidewire 170. The guidewire sheath 172 can be formed of a thin, gas impervious film material, or the like, and can be configured to enclose the guidewire 170 and prevent contamination with pathogens or the like.

As shown in FIG. 3D, with the guidewire 170 extended into the vasculature, the anchor 120 can be removed from the insertion site 72 to provide a clear pathway for the catheter 150 to advance over the guidewire 170. To remove the anchor 120, the clinician can retract the anchor hub 130 proximally. In an embodiment, the anchor hub 130 is slidably engaged with the housing 110 along a longitudinal axis. In an embodiment, the anchor hub 130 can be coupled to the syringe barrel 142. As such, a clinician can continue to withdraw the syringe barrel 142 in a single motion to remove the needle 102 and the anchor 120.

FIGS. 4A-4C show further details of the retraction and splitting of the anchor 120. The first arm 132A and the second arm 132B can be formed of a resilient material and can flex radially outward from a central axis 30 to pass either side of the cleaver portion 112. Each arm 132 can be attached to a portion of the anchor 120, for example, a first arm 132A can be attached to a first portion 120A and the second arm 132B can be attached to a second portion 120B. As such, urging the anchor hub 130 proximally with one hand, can urge the arms 132A, 132B radially outward, causing the anchor 120 to separate into the first portion 120A and the second portion 120B. In an embodiment, the anchor 120 can include a tear line 122, as described herein, configured to facilitate separation of the anchor 120. In an embodiment, the cleaver 112 can include a sharpened distal edge configured to facilitate separation of the anchor 120. Separation of the anchor 120 into the first portion 120A and the second portion 120B can provide a clear pathway for the catheter 150 to pass therebetween. Advantageously, retraction of the anchor hub 130 and splitting of the anchor sheath 120 can be performed with a single hand, freeing the other hand to stabilize the insertion site or allowing the user more control over the device as a whole.

As shown in FIG. 3E, with the anchor 120 split and removed from the vasculature, the clinician can advance the catheter 150 using the catheter advancement assembly 160. The catheter 150 can be advanced over the guidewire 170 until a distal tip 154 of the catheter 150 is disposed at a target location within the vasculature. To note, for ease of illustration, FIG. 3E shows only the housing 110, catheter 150, catheter advancement assembly 160 and guidewire 170 of the catheter placement system 100.

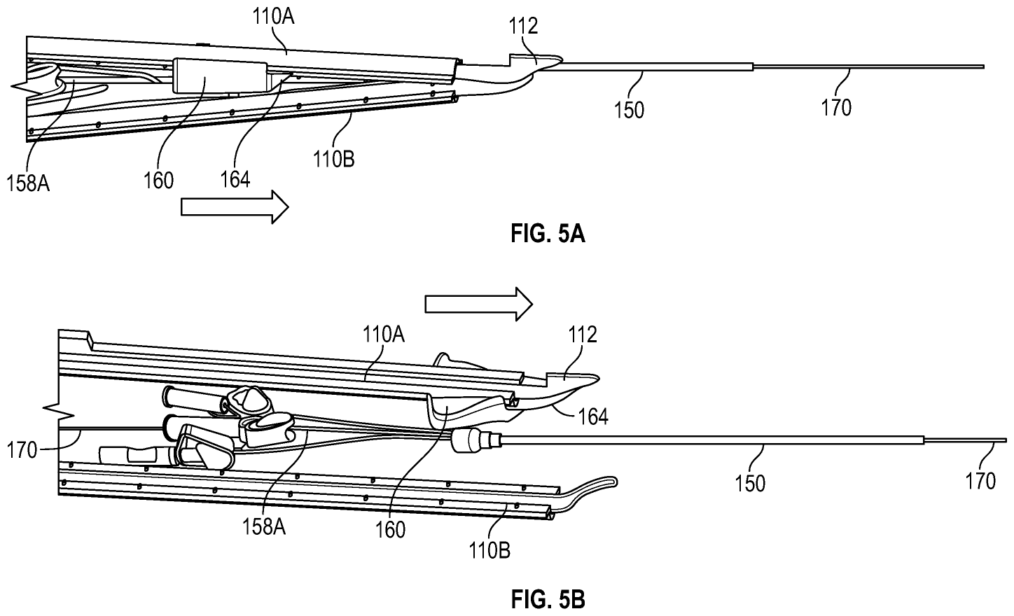

In an embodiment, advancing the catheter advancement assembly 160 can urge a distal edge of the catheter advancement assembly 160 between a first housing portion 110A and a second housing portion 110B. FIGS. 5A-5B show further details of the catheter advancement assembly 160 separating the first housing portion 110A and a second housing portion 110B. In an embodiment, the catheter advancement assembly 160 can be slidably engaged with a lower surface of the first housing portion 110A. A second housing portion 110B can be releasably secured to a lower surface of the first housing portion 110A and configured to encase a portion of the catheter 150 therebetween to secure the catheter 150 within the housing 110. The second housing portion 110B can be releasably secured to the first housing portion 110A using adhesive, bonding, welding, ultrasonic welding, one or more frangible bridges, interference fit, snap fit or press fit engagements, combinations thereof, or the like. A distal portion 164 of the catheter advancement assembly 160 can define a wedge shape. As the catheter advancement assembly 160 is advanced distally, the wedge-shaped distal portion 164 can be urged between the first housing portion 110A and the second housing portion 110B to separate the housing 110 and release the catheter 150 therefrom. With the catheter 150 positioned within the vasculature the housing portions 110A, 110B can be separated and removed and the guidewire 170 can be withdrawn proximally from the catheter 150.

Advantageously, embodiments of the catheter placement system 100 can access the vasculature and confirm correct vascular access using the needle and anchor assembly, having a relatively thin diameter. Should the vascular access be incorrect, closing the insertion site 72 can be relatively easy by applying pressure, and the access can be reattempted. If the vascular access is correct, the anchor 120 is already in place to maintain patency of the insertion site 72. Only the soft, compliant anchor 120 need be advanced, leaving the needle 102 in place. Once the anchor 120 has been advanced the needle 102 can be removed without risking any trauma to the vessel. As such the process of accessing and anchoring the insertion site 72 is expedited. Further the anchor 120 does not obstruct the needle lumen 106 further expediting the placement process. Advantageously, splitting and removing the anchor 120 can be performed concurrently with removal of the needle 102 reducing the steps involved, and simplifying and expediting the placement process yet further. Further, all of the components for placing the catheter 150 can be contained within a single sterile environment, preventing direct contact and mitigating the introduction of pathogens and other infectious causing agents.

Advantageously, since the anchor 120 is disposed on an outer surface of the needle, it does not obstruct the pathway of the guidewire 170 through a lumen of the needle 102. As such, the guidewire 170 can be “pre-loaded” within the catheter 150 and optionally within a portion of the needle 102. This can provide a longitudinally more compact system 100, shortening the advancement distance, and providing a more convenient and easier to use system. This can be of particular importance since a length of the guidewire doubles with the length of the catheter 150.

Additionally, with more of the guidewire disposed within the catheter placement system 100, the system 100 can provide more columnar support to the guidewire 170 when advancing the guidewire into the vasculature, mitigating collapsing or kinking of the guidewire 170. As such the clinician can manipulate from a proximal end 176 of the guidewire without needing to touch a mid-portion of the guidewire 170 that may be inserted into the patient. The guidewire 170 can further include a guide wire hub coupled to the proximal end 176, preventing the guidewire from being accidentally drawn entirely into the vasculature. Further the catheter placement system mitigates the reintroduction of tools to/from the insertion site 72, saving time and mitigating the introduction of pathogens.

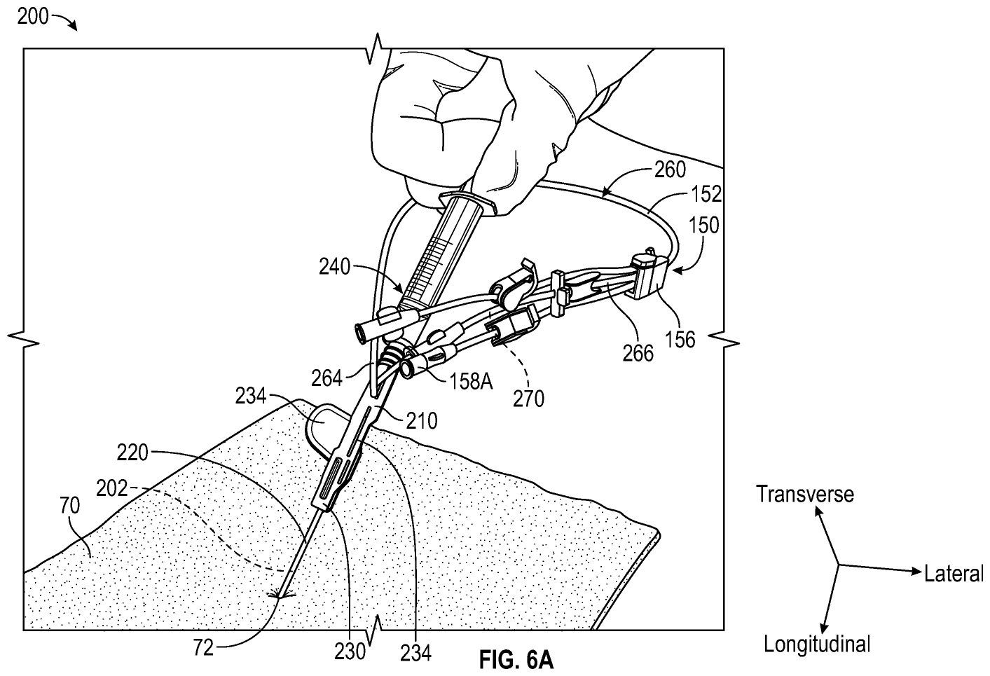

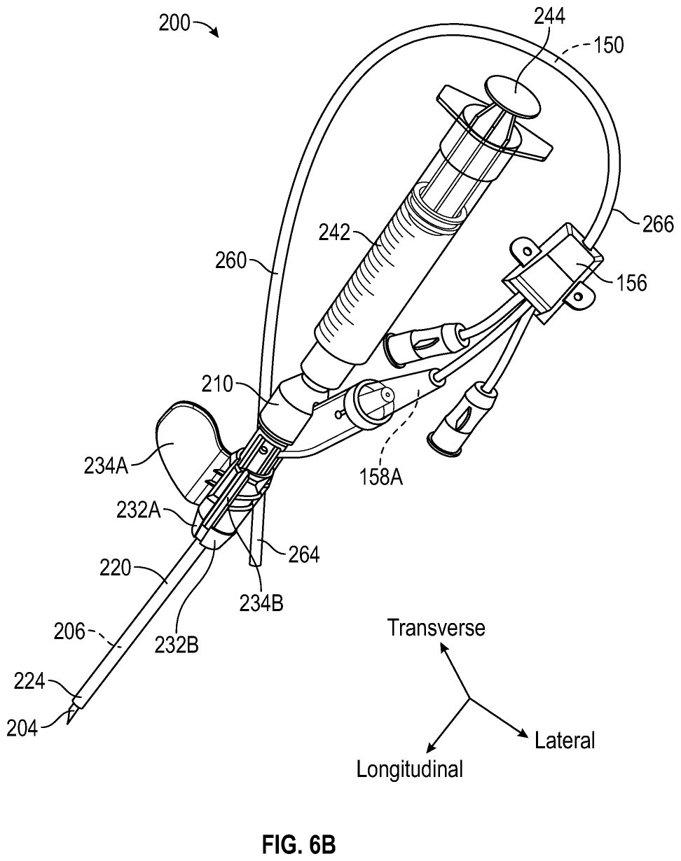

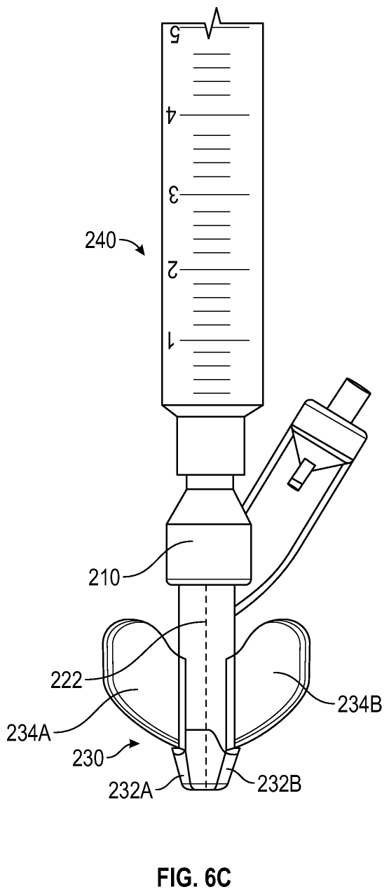

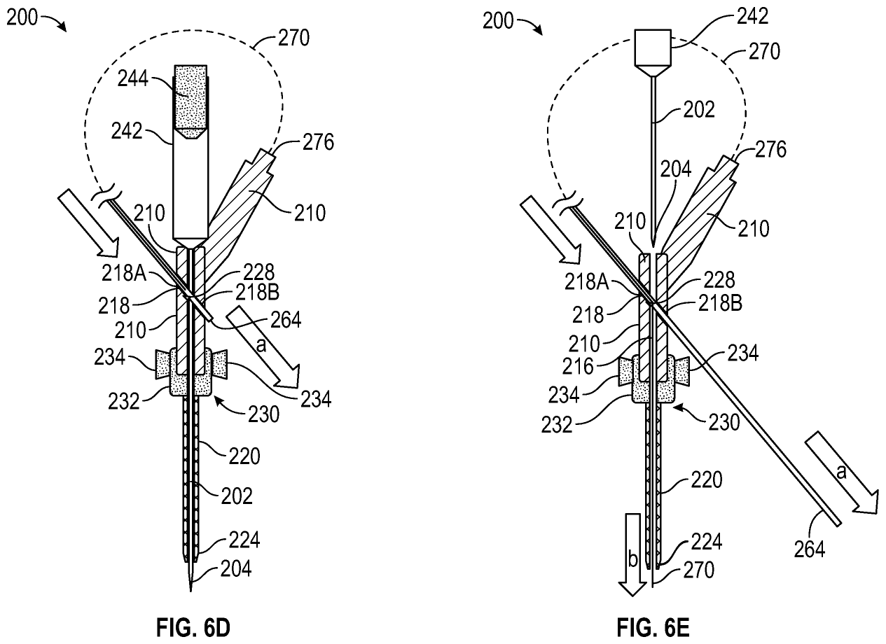

FIGS. 6A-6E show various details of a catheter placement system 200. FIG. 6A shows a perspective view of an embodiment of a catheter placement system 200 generally including housing 210 defining a lumen 216 extending longitudinally therethrough. A needle 202 can be supported by a syringe barrel 242 of a flashback assembly 240 and can extend through the lumen 216 of the body 210 and distally of the housing 210. An anchor hub 230 can be coupled to a distal end of the housing 210 and can support a splittable anchor sheath 220 slidably engaged with an outer surface of the needle 202. The catheter placement system 200 can further include a guidewire 270 pre-loaded within a catheter 150 which is contained within a splittable advancement sheath 260. In an embodiment, a proximal end 266 of the advancement sheath 260 can be recurved back onto, and coupled with, the housing 210. In an embodiment, a proximal end of a first extension leg 158A of the catheter 150 can be recurved back onto, and coupled with, the housing 210.

FIGS. 6B-6E show further details of the catheter placement system 200. The needle 202 can extend along a longitudinal axis and include a sharpened distal tip 204 configured for piercing a skin surface 70 at an insertion site 72 and access a vasculature of a patient. The needle 202 can define a needle lumen 206 configured to provide fluid communication between the distal tip 204 and a flashback assembly 240. A proximal end of the needle can be supported by syringe barrel 242. The needle 202 can be slidably engaged with the housing 210 through a housing lumen 216 that extends along a longitudinal axis.

The flashback assembly 240 can include a syringe barrel 242 in fluid communication with the needle lumen 206, and a plunger 244 slidably engaged with the syringe barrel 242. Sliding the plunger 244 proximally relative to the syringe barrel 242 can create a vacuum therein which can cause a proximal fluid flow through the needle lumen 206 and into to the syringe barrel 242. In an embodiment, a portion of the syringe barrel 242 can be formed of a transparent material configured to allow observation of a blood flow therethrough. As such, a clinician can observe a color and/or pulsatile flow to confirm correct vascular access.

The catheter placement system 200 can further include a splittable anchor sheath 220 disposed annularly about an outer surface of the needle 202 and slidably engaged therewith. The anchor 220 can define a substantially elongate tubular shape with a tapered distal tip 224. The distal tip 224 can fit tightly about the needle 202 to allow the needle 202 and anchor 220 assembly to be inserted through the insertion site 72 and into a vasculature of a patient. In an embodiment, the anchor 220 can be supported at a proximal end by an anchor hub 230 configured to slide the anchor 220 relative to the needle 202. In an embodiment, a distance between the distal tip 204 of the needle 202 and the distal end of the anchor hub 230 can be between 5 cm and 10 cm long. However, it will be appreciated that greater or lesser distances are also contemplated. In an embodiment, a distance between the distal tip 204 of the needle 202 and the distal end of the anchor hub 130 can be 7 cm long.

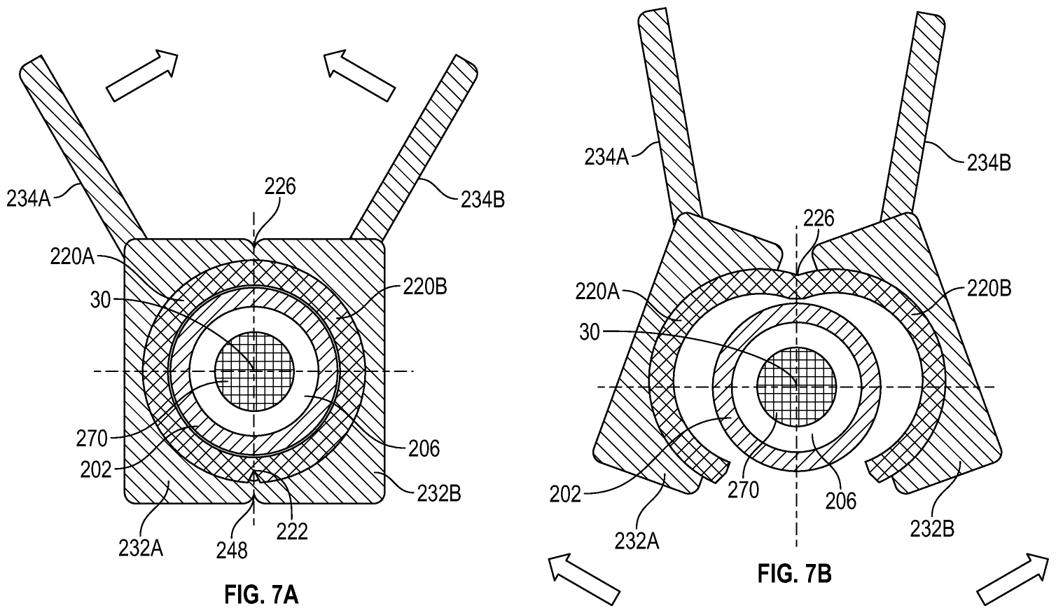

In an embodiment, as shown in FIGS. 7A-7B, the splittable anchor 220 can include one or more tear lines 224 extending longitudinally along an outer surface thereof. The tear line 222 can include a groove, score-line, perforation, laser cut line, or similar line of weakness extending longitudinally and extending at least partially into a wall of the anchor 220. The tear line 222 can be configured to facilitate separation of two or more portions of the anchor 220 to allow removal of the anchor 220.

In an embodiment, the anchor hub 230 can include an arm 232 extending distally. A distal portion of the arm 232 can be coupled with a proximal portion of the anchor 220. As shown in FIGS. 6A-7B, the anchor hub 230 can include a first arm 232A coupled to a first portion 220A of the anchor 220 and a second arm 232B coupled to a second portion 220B of the anchor 220. In an embodiment, each arm 232 can further include a wing 234, extending radially therefrom, for example a first arm 232A can include a first wing 234A and a second arm 232B can include a second wing 234B. In an embodiment, the first arm 232A can be hingedly rotatable relative to the second arm 232B about a fulcrum 226 disposed opposite the tear line 222, across the central axis 30. Each of the wings 234A, 234B can extend from the respective arm 232A, 232B across the fulcrum 226.

In use, a clinician can pinch the wings 234A, 234B together along an axis extending perpendicular to the longitudinal axis, e.g. the lateral axis, causing the arms 232A, 232B to hingedly rotate relative to each other about the fulcrum 266. This in turn can cause a first portion of the anchor 220 coupled to first arm 232A to separate from a second portion of the anchor 220 coupled to the second arm 232B, along the tear line 222 (FIG. 7B). The anchor 220 can then be peeled away from the needle 202 and/or guidewire 270 disposed therein, as described in more detail herein. In an embodiment, the first arm 232A and the second arm 232B can be formed integrally to fully encircle the anchor sheath 220 about the longitudinal axis. Further, the first arm 232A can be coupled to the second arm 232B along a separation line 248, or similar line of weakness, aligned adjacent to the tear line 222 of the anchor 220 and configured to facilitate separation of the first arm 232A from the second arm 232B, as described herein. In an embodiment, the anchor hub 230 can be slidably engaged with the needle 202 and configured to either advance the anchor 220 distally, or withdraw the anchor 220 proximally relative to the needle 202.

In an embodiment, the system 200 can further include a catheter 150 such as a multi-lumen central venous catheter (“CVC”), as described herein. However, it will be appreciated the CVC catheter 150 is a non-limiting example, and that embodiments disclosed herein can be used with various single lumen or multi-lumen elongate vascular access devices such as peripherally inserted central catheter (PICC), rapidly insertable central catheters (RICC), dialysis catheters, and the like.

The catheter 150 can generally include an elongate body 152 defining one or more catheter lumen. A distal tip 154 of the catheter body 152 can include an opening communicating with a lumen. A proximal end of the body 152 can be supported by a catheter hub 156. In an embodiment, the catheter 150 can further include one or more extension legs 158 extending proximally from the catheter hub 156. Each extension leg can communicate with a lumen of the catheter body 152. In an embodiment, the catheter body 152, the catheter hub 156, extension leg 158, or combinations thereof, can be enclosed within a splittable advancement sheath 260. The advancement sheath 260 can allow a clinician to manipulate the catheter 150, without directly contacting the catheter 150 and while maintaining the catheter 150, or portions thereof, within a sterile environment. In an embodiment, the advancement sheath 260 can be formed of a compliant material such as a plastic, polymer, elastomer, or the like.

In an embodiment, the system 200 can further include a guidewire 270 disposed within a lumen of the catheter 150. In an embodiment, a distal end of the advancement sheath 260 can be coupled with the housing 210 and can align the catheter 150 with a channel 218 of the housing 210. In an embodiment, a proximal end 266 of the advancement sheath 260 can be recurved towards the housing 210. In an embodiment, a proximal end 266 of the advancement sheath 260 can be coupled to the housing 210. In an embodiment a proximal end of a first extension leg 158A can be releasably coupled to the housing 210 to enclose a guidewire 270, disposed therein, in a sealed environment.

As shown in FIGS. 6D-6E, the housing 210 can include a lumen 216 extending along a longitudinal axis, and a sheath channel 218 intersecting the lumen 216 at an angle relative to an axis the lumen 216. A distal portion 264 of the advancement sheath 260 containing one or both of the guidewire 270 and the catheter 150 therein, can extend through the channel 218 from a first opening 218A disposed on a first side of the housing 210, to a second opening 218B, disposed on a second side of the housing 210, opposite the first side. In an embodiment, a distal portion of the guidewire 270 and/or the catheter body 152 can extend through the first opening 218A of the channel 218 and extend into the housing lumen 216.

At the junction between the channel 218 and the housing lumen 216, the advancement sheath 260 can split to allow the distal portion of the guidewire 270 and/or the catheter body 152 to exit the advancement sheath 260 and enter the housing lumen 216, while the corresponding portion of the advancement sheath 260 can exit the housing 210 through the channel 218 at the second channel opening 218B. In an embodiment, the housing can further include a blade 228 disposed at a junction between the channel 218 and the housing lumen 216 and configured to split the advancement sheath 260 axially, as the advancement sheath 260 is pulled through the channel 218. In an embodiment, the advancement sheath 260 can include a tear line 262, or similar line of weakness as described herein, extending axially along the advancement sheath 260 and configured to facilitate separation of the advancement sheath 260 to allow one of the guidewire 270 or the catheter body 152 to enter the housing lumen 216. Advantageously, the blade 228 and/or tear line 262 can be configured to reduce any friction or drag during the splitting process.

In use, a clinician can grasp a distal portion 264 of the advancement sheath 260 and pull the advancement sheath 260 through the channel 218 along a first direction (a) at an angle relative to the longitudinal axis. In doing so, the guidewire 270 and/or catheter 150 can detach from the advancement sheath 260 and advance along the lumen 216 in a second direction (b) substantially parallel to the longitudinal axis. Advantageously, the clinician can release the guidewire 270 and/or catheter 150 from the advancement sheath 260 and advance along the housing lumen 216 into the vasculature without directly contacting the guidewire 270 or the catheter 150.

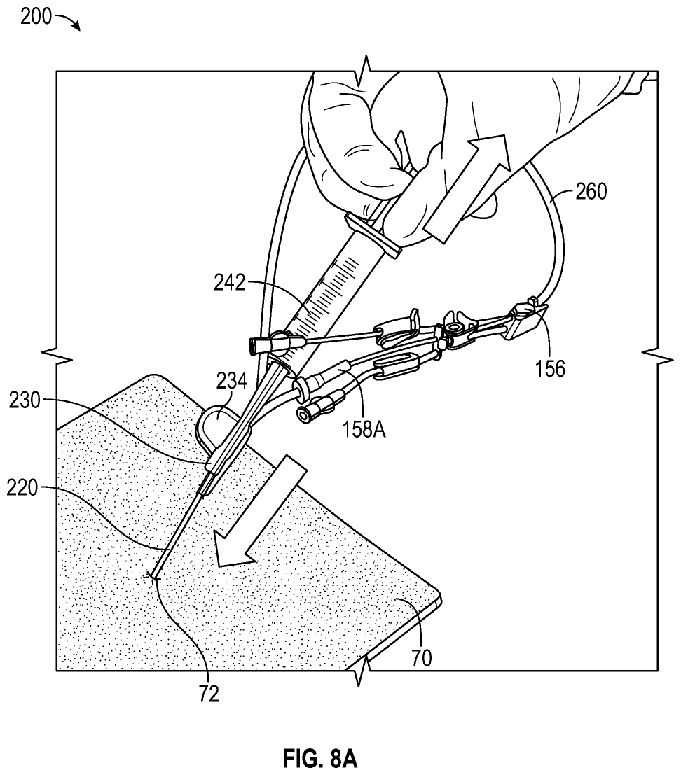

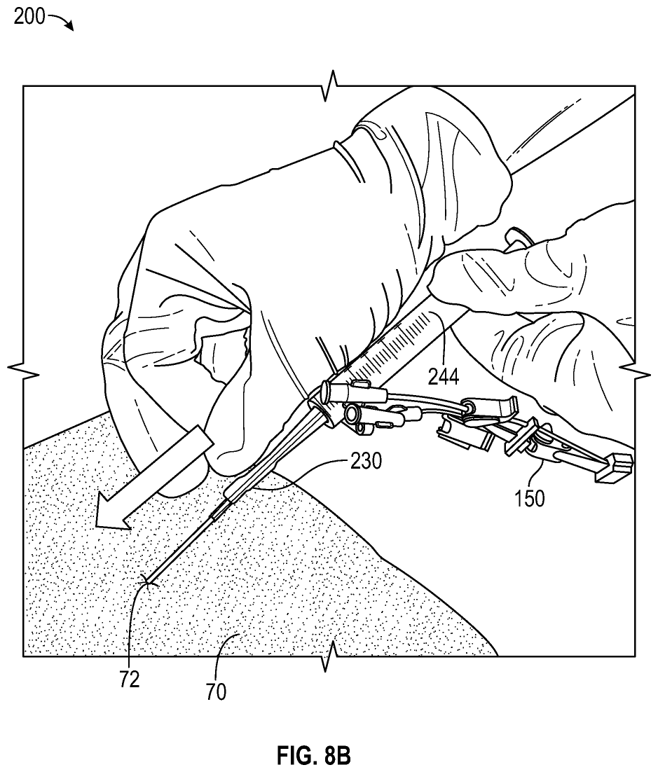

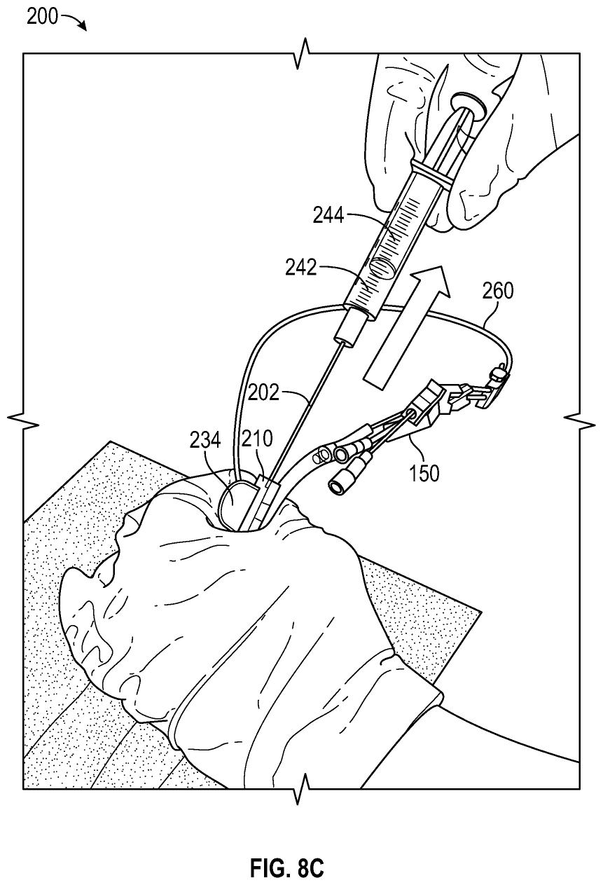

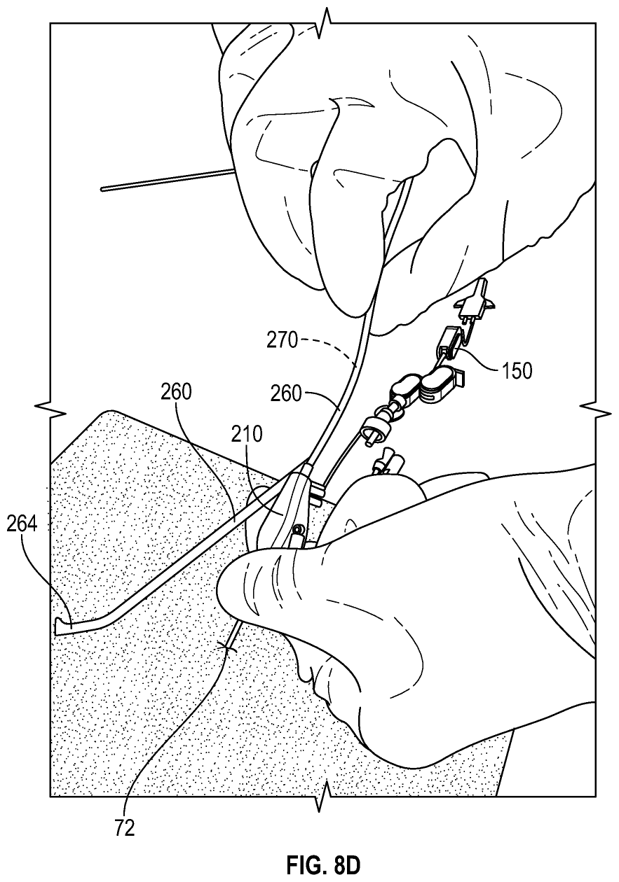

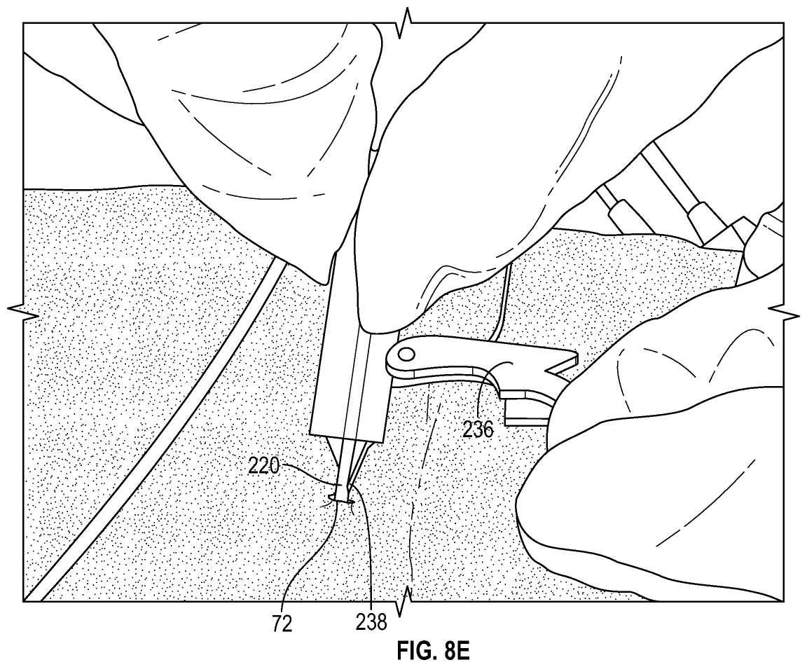

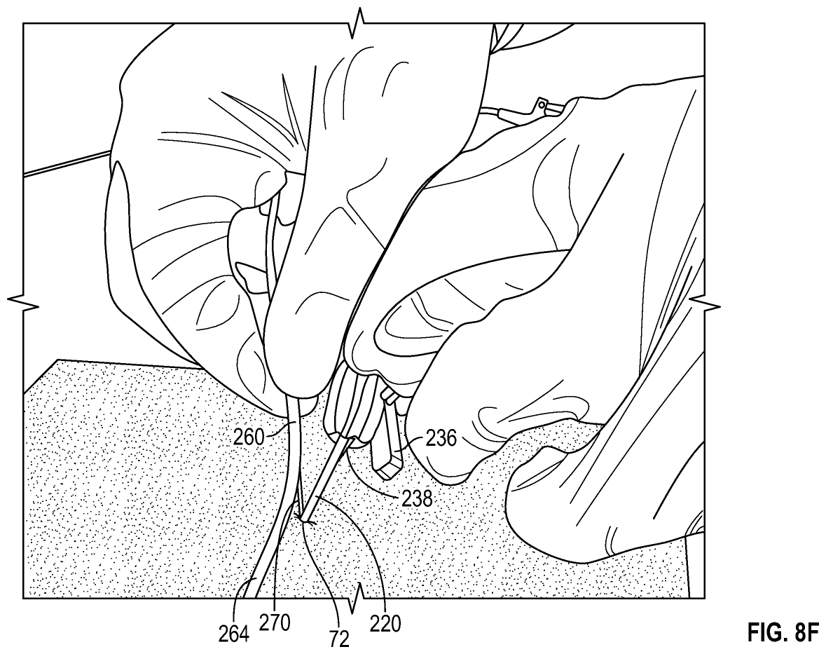

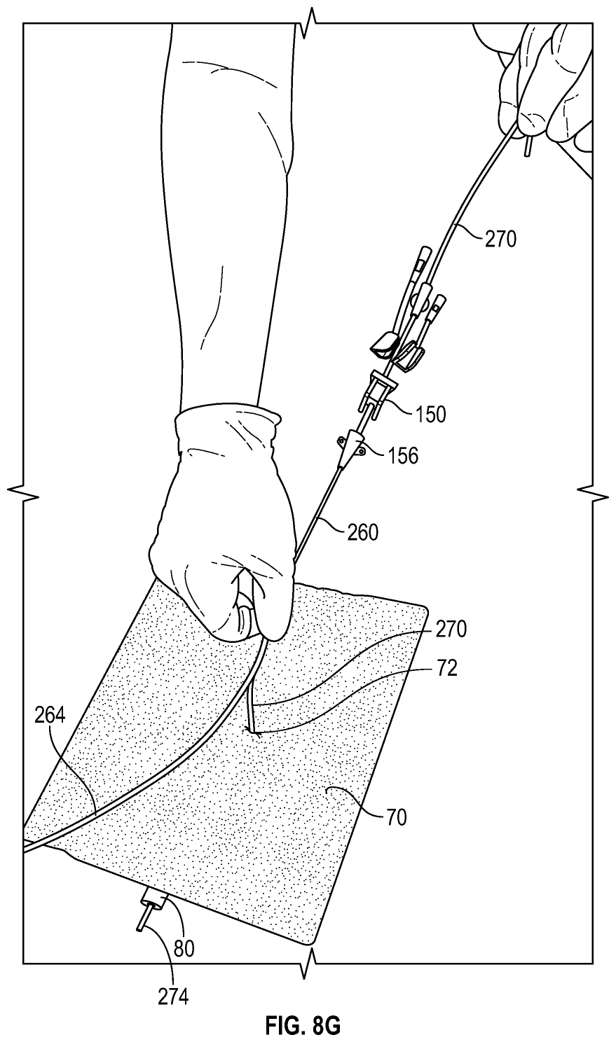

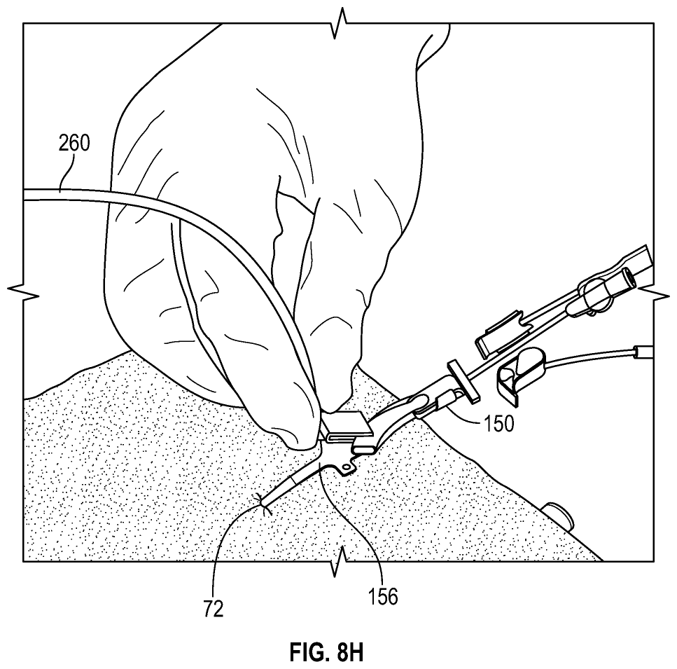

In an exemplary method of use, as shown in FIGS. 8A-8H, a catheter placement device 200 is provided as described herein. A clinician can access a vasculature of a patient with the needle 202 by manipulating one of the housing 210 or the syringe barrel 242. The clinician can withdraw the plunger 244 and observe a proximal blood flow to confirm correct vascular access. As shown in FIG. 8B, with the needle tip 204 and distal end 224 of the anchor 220 disposed within the vasculature, the clinician can grasp the anchor hub 230, e.g. by a wing 234, and advance the anchor 220 over the needle 202 and into the vasculature. The anchor 220 can maintain patency of the insertion site 72. The position of the needle 202 relative to the insertion site 72 can substantially remain undisturbed as the anchor 220 is advanced. In an embodiment, the anchor hub 230 and the housing 210 assembly can be advanced over the needle 202 while the syringe barrel 242 and the needle 202 coupled thereto remain substantially stationary relative to the insertion site 72.

As shown in FIG. 8C, once the anchor 220 can been advanced into the vasculature, the needle 202 can be withdrawn from the insertion site 72 by withdrawing the syringe barrel 242 and needle 202 assembly proximally from the housing lumen 216. In an embodiment the syringe barrel 242 and needle 202 can be detached from the housing 210 and discarded. In an embodiment, a needle tip 204 can be locked within the housing 210 in a needle safety mechanism.

As shown in FIG. 8D, once the needle 202 has been removed from the housing lumen 216, the clinician can then advance the guidewire 270 through the channel 218, through the housing lumen 216, and into the lumen of the anchor 220. In an embodiment, to advance the guidewire 270, the clinician can urge the advancement sheath 260, including the guidewire 270 disposed therein, through the channel 218. As the advancement sheath 218 passes through the junction between the channel 218 and the housing lumen 216, the advancement sheath 260 can split, releasing the guidewire 270 therefrom. The advancement sheath 260 can then continue through the channel 218 and the guidewire 270 can enter the housing lumen 216.

In an embodiment, to advance the guidewire 270, the clinician can detach a proximal end of the first extension leg 158A from the housing 210 and manipulate a proximal end 276 of the guidewire 270 extending therefrom to advance the guidewire 270. The guidewire 270 can advance through the lumen of the catheter 150, disposed within the advancement sheath 260, through the channel first opening 218A and enter the housing lumen 216. The clinician can then advance the guidewire 270 until a distal tip is disposed at a target location within the vasculature.

In an embodiment, as shown in FIG. 8E, once the guidewire 270 has been advanced to the target location the clinician can perform a skin nick incision at the insertion site 72 to allow for the introduction of the catheter 150 through the insertion site 72. As will be appreciated, the diameter of the catheter 150 can be larger than the diameter of the needle 202 and anchor 220. In an embodiment, the anchor hub 230 can include a blade 238 disposed proximate a distal end of the anchor hub 230. A clinician can remove a blade cover 236 and advance the anchor hub 230 along the guide wire 270 until the blade 238 enters and incises the insertion site 72.

As shown in FIG. 8F, with the guidewire 270 in place, the anchor 220 can then be split and removed. The clinician can grasp the wings 234 and pinch them together, pivoting the arms 232 about the fulcrum 226 to separate a proximal portion of the anchor 220 along the tear line 222A releasing the guidewire 270 therefrom. The anchor 220 and anchor hub 230 can then be withdrawn proximally from the insertion site 72, leaving the guidewire 270 in place.

As shown in FIG. 8G, once the anchor 220 has been removed, the catheter 150 can be advanced over the guidewire 270 and into the vessel 80. In an embodiment, to advance the catheter 150, the clinician can detach a proximal end of the first extension leg 158A from the housing 210 and grasp a distal end 264 of the advancement sheath 260. The clinician can then pull the distal end 264 of the advancement sheath 260 through the channel 218 of the housing 210. The advancement sheath 260 can be configured to split within the housing 210 at the junction of the channel 218 with the housing lumen 216 releasing the catheter 150 and allowing the catheter to advance through the housing lumen 216 and into the vasculature.

In an embodiment, the wings 234 of the anchor hub 230 can be configured to split both the anchor 220 along a tear line 222 as well as the housing 210 along a separation line. As such, the anchor 220 can be split and removed as described herein. Further, the housing 210 can also be split and removed, leaving the guidewire 270 disposed in the insertion site and the catheter 150, disposed within the advancement sheath 260, slidably engaged with the guidewire 270.

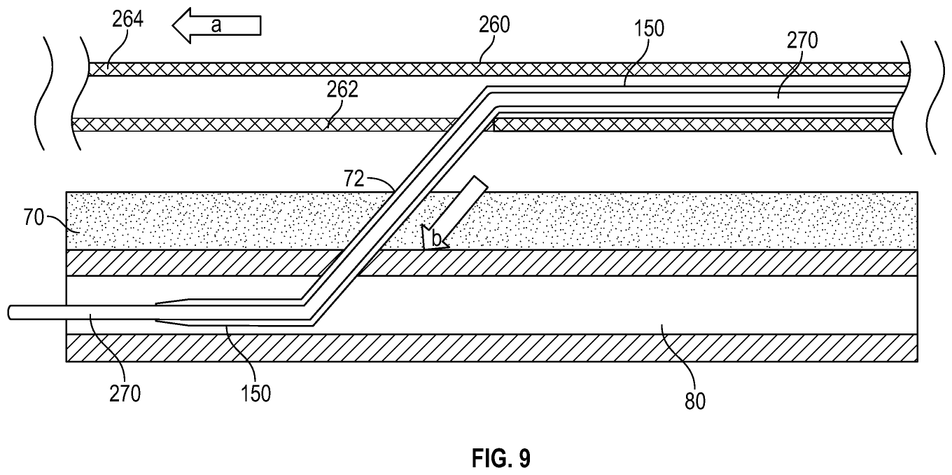

As shown in FIG. 9, to advance the catheter 150, the clinician can grasp a distal portion 264 of the advancement sheath 260 and pull in a first direction (a) pulling the catheter 150, which is disposed within the advancement sheath 260, along with it. When a wall of the advancement sheath 260 including the tear line 262 meets the guidewire 270 extending into the insertion site 72, the guidewire can split the advancement sheath 260 axially along the tear line 262. As the advancement sheath 260 splits, the catheter 150 can be urged along the guidewire 150 and into the insertion site 72 accessing the vasculature. As shown in FIG. 8H, once a distal tip of the catheter 150 has been advanced to the target location the remainder of the advancement sheath 260 can be split and removed from the catheter 150, and the hub 156 secured to the skin surface. The guidewire 270 can then be withdrawn proximally from the lumen of the catheter 150.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter placement system, comprising:
a housing;
a flashback assembly slidably engaged with the housing;
a needle supported by the flashback assembly;
a catheter supported by a catheter advancement assembly that is slidably engaged with the housing;
an anchor sheath slidably engaged with an outer surface of the needle, a portion of the anchor sheath configured to be advanced distally of the needle to anchor an incision site; and
an anchor hub configured to split the anchor sheath axially and remove the anchor sheath proximally.

2. The catheter placement system according to claim 1, wherein the anchor sheath is formed of a compliant material including one of a plastic, polymer, PTFE, or an elastomer.

3. The catheter placement system according to claim 1, wherein the catheter includes one of a CVC catheter, a rapidly insertable central catheter, or a PICC catheter.

4. The catheter placement system according to claim 1, wherein the flashback assembly includes a syringe barrel in fluid communication with a lumen of the needle and a plunger slidably engaged therewith, the flashback assembly configured to draw a blood flow through the needle lumen.

5. The catheter placement system according to claim 1, further including a guidewire disposed within a lumen of the catheter.

6. The catheter placement system according to claim 1, wherein the anchor hub includes an arm configured to engage a cleaver portion of the housing and configured to deflect the arm radially outward.

7. The catheter placement system according to claim 1, wherein the anchor hub includes a first arm coupled to a first portion of the anchor sheath and a second arm coupled to a second portion of the anchor sheath.

8. The catheter placement system according to claim 7, wherein the anchor sheath includes a tear line extending axially therealong and configured to allow the first portion of the anchor sheath to separate from the second portion of the anchor sheath.

9. The catheter placement system according to claim 1, wherein the tear line includes one of a groove, a score-line, a perforation, or a laser cut line.

10. A method of placing a catheter, comprising:
accessing a vasculature with a distal tip of a needle and a distal tip of an anchor sheath assembly, the anchor sheath disposed on the needle;
confirming vasculature access with a flashback assembly in fluid communication with the needle;
sliding the distal tip of the anchor sheath distally of the distal tip of the needle into the vasculature;
removing the needle proximally from the vasculature;
advancing a guidewire through a lumen of the anchor sheath into the vasculature;
splitting the anchor sheath along a longitudinal axis and removing the anchor sheath proximally;
splitting a housing with a catheter advancement assembly to release a catheter therefrom; and
advancing a catheter over the guidewire into the vasculature.

11. The method according to claim 10, wherein splitting the anchor sheath includes withdrawing an anchor hub proximally over a cleaver portion, the cleaver portion splitting the anchor sheath along a tear line.

12. The method according to claim 10, wherein the anchor hub includes a first arm coupled to a first portion of the anchor sheath, and a second arm attached to a second portion of the anchor sheath, each of the first arm and the second arm deflecting radially outward from a central axis to split the anchor sheath along the longitudinal axis.

13. The method according to claim 10, wherein confirming vasculature access further includes withdrawing a plunger from a syringe barrel of the flashback assembly, and drawing a blood flow proximally through the needle and observing a blood flow.

14. The method according to claim 10, wherein removing the needle proximally from the vasculature includes sliding the flashback assembly proximally, the needle coupled to the flashback assembly.

15. The method according to claim 10, wherein advancing the catheter includes advancing a catheter advancement assembly supporting the catheter, the catheter assembly separating a first housing portion from a second housing portion to disengage the catheter from the housing.

16. A catheter placement device, comprising:
a housing defining a housing lumen extending along a longitudinal axis and a channel extending at an angle relative to the longitudinal axis and intersecting the housing lumen;
a needle supported by a syringe and configured to extend through the housing lumen;
an anchor sheath supported by an anchor hub that is coupled to a distal end of the housing, the anchor sheath slidably engaged with the needle; and
a catheter including a guidewire disposed within a lumen of the catheter and contained within an advancement sheath, a distal end of the advancement sheath configured to extend through the channel, one or both of the guidewire and the catheter configured to separate from the advancement sheath within the channel and extend through the housing lumen.

17. The catheter placement device according to claim 16, wherein the advancement sheath includes a tear line extending along an axial length and configured to allow a first portion of the advancement sheath to separate from a second portion of the advancement sheath to allow one of the guidewire or the catheter to separate from the advancement sheath.

18. The catheter placement device according to claim 16, wherein the housing further includes a blade disposed proximate a junction between the channel and the housing lumen and configured to cut through a wall of the advancement sheath as the advancement sheath is urged through the channel.

19. The catheter placement device according to claim 16, wherein the anchor hub includes a separation line configured to facilitate separation therealong and to split the anchor sheath along a tear line extending axially along the anchor sheath.

20. The catheter placement device according to claim 16, wherein the anchor hub further includes a first wing and a second wing hingedly rotatably relative to each other and configured to separate a first portion of the anchor hub from a second portion of the anchor hub.

21-27. (canceled)
